(19) United States
(12) Patent Application Publication
(10) Pub. No.: US 2007/0179084 A1
(43) Pub. Date: Aug. 2, 2007

(54) Process for reducing the concentration of blood glucose in a diabetic patient

(75) Inventors: Cheng Chang, Taipei City (TW); Ying-Nan Lin, Keelung City (TW)

Correspondence Address: RABIN & Berdo, PC, 1101 14TH STREET, NW, SUITE 500, WASHINGTON, DC, 20005, US

(73) Assignee: Nation Chiao Tung University, Hsinchu (TW)

(21) Appl. No.: 11/723,101

(22) Filed: Mar. 16, 2007

Related U.S. Application Data
(63) Continuation-in-part of application No. 11/341,482, filed on Jan. 30, 2006.

(51) Int. Cl. A61K 38/40

(52) U.S. Cl. 514/6

(57) ABSTRACT

A complex for serving diabetics to reduce the concentration of blood glucose is provided, including a transferrin and a metal ion (or an oxidized metal ion). The transferrin can carry the metal ion (or the oxidized metal ion) into cells of a patient, thereby encouraging glucose uptake by the cells and reducing the toxicity of the metal ion (or the oxidized metal ion). Furthermore, the complex has the capability of reducing insulin resistance, and therefore is available to treat high blood glucose in type 2 diabetes and reduce insulin resistance. A process for reducing blood glucose of a diabetic patient includes providing a composition comprised of a complex of transferrin and at least one metal ion. A dose of the composition is administered to the diabetic patient in an amount effective to encourage glucose uptake by the cells and reduction of glucose in the blood as the transferrin carries the metal ion (or the oxidized metal ion) into the cells of the diabetic patient.

### CROSS-REFERENCE TO RELATED APPLICATION

This is a continuing application of applicants' application Ser. No. 11/341,482, filed Jan. 30, 2006.

### BACKGROUND

1. Field of Invention

The invention relates to a complex for treating diabetes, and more particularly to a complex for serving diabetics to reduce the concentration of blood glucose, which includes a transferrin and a metal ion (or an oxidized metal ion).

2. Related Art

Diabetes is a disorder of the metabolism, resulting from the fact, that the body of the diabetic is unable to make normal use of glucose as fuel because of a failure of insulin secreted by the beta cells of the pancreas or an impaired capability of insulin in normal insulin secretion. Type 1 diabetes is an autoimmune disease, meaning that the body stages an all-out war against itself by attacking its own cells. The immune system of the diabetic can attack and destroy its own pancreatic beta cells, which secrete the insulin, and then the end result is the inability of the pancreas to produce the hormone insulin. Concerning patients with type 2 diabetes, the pancreas usually produces enough insulin, but the bodies are unable to respond to the produced insulin because the bodies resist to the action of this insulin and their pancreatic beta cells have dysfunction.

In the present, two classes of a therapy for diabetes are the drug therapy and the non-drug therapy. The non-drug therapy includes nutrition, -exercise and formal diabetes education. The exercise can cause muscles to shrink, such as to stimulate the glucose transporter 4 (GLUT4) to be translocated to the cell membrane, and therefore encourage glucose uptake. This mechanism is different from that responsible for the activation for cellulous absorption by the insulin, and is unimpaired concerning patients with type 2 diabetes. The main object of drug therapy is the rise of insufficient insulin, reduction of high blood glucose after eating, and the improvement of the insulin resistance. Consequently, the therapies and the drugs include the therapies related to the insulin, oral hypoglycemic agents (OHA), and a combined drug therapy. The two classes of drugs for treating diabetes are Sulphonylureas (SU) and Biguanides (BG). The SU can stimulate the pancreas to secrete the insulin, and more particularly enhance the function that pancreatic beta cells secrete insulin in response to the glucose. The BG cannot stimulate insulin secretion by itself, but the mechanism thereof regulating the blood glucose inhibits the appetite, postponing gut absorbing glucose, promoting anaerobic decomposition of glucose in the gut to increase the use of glucose therein, enhancing the action of the insulin on the liver, and promoting the GLUT4 stored in the cells to be translocated to the cell membrane for transportation. Therefore, the glucose metabolism is accelerated. But, after the blood-glucose-lowering drugs mentioned above are treated for a long time, the property of the drugs is lost.

It is known that diabetes is involved with metal ions. For example, a zinc ion can activate the activity of insulin. Cobalt (II) and cobalt (III) dipicolinate complexes have the same activity as insulin, but these dipicolinate complexes may constantly accumulate within the body and then cause side effects. Further, it is discovered that diabetics have less chromium ions in their body than normal persons. So a suitable supplement of chromium ions is conducive to the regulation of blood glucose of the diabetics. The mineral chromium ingested in their regular diet includes an inorganic chromium and organic chromium compound. The absorption of inorganic chromium is very low, merely from 0.3% to 0.4%, because olation easily occurs in the digestive tract to form huge dipicolinate complexes, obstructing absorption in the gut. U.S. Pat. No. 6,379,693 describes a complex compound of trivalent chromium and lactoferrin. In use, the complex compound is added to milk, thereby reducing the toxicity of the metal ions and facilitating the absorption in the gut. Since lactoferrin can resist digestion with gastrointestinal proteases and the metal ions are released at pH of about 3.5, the lactoferrin can effectively carry the metal ions into gastrointestinal cells. After the lactoferrin enters the gastrointestinal cells by endocytosis, it is decomposed by lysosome and the metal ions are released into the blood to be carried into cells, where the metal ions are used, by transferrin. Hence, when the chromium ions are carried into other organs/tissues via the circulatory system, they're not in the mode of the complex compound of trivalent chromium and lactoferrin. Moreover, if the metal ions are directly used for treatment, they constantly accumulate within the body, such as causing metal intoxication. The transferrin is a protein carrying an iron ion, and is synthesized by hepatocytes. In blood, the content of the transferrin is 2.5 milligrams per milliliter (mg/ml). Transferrin exists in blood, bile, amniotic fluid, cerebrospinal fluid, the lymph, colostrums and milk. Transferrin can bind with metal ions, such as iron (III) ions (Fe³⁺), copper (II) Ions (Cu²⁺), zinc (II) ions (Zn²⁺), manganese (III) ions (Mn³⁺), cobalt (III) ions (Co³⁺), vanadium (III) ions (V³⁺), chromium (III) ions (Cr³⁺), etc., and their binding positions are the same. The complex of the transferrin and the iron ion can bind to a transferrin receptor on cell membrane, and then enter into the cell by the endocytosis to form endosome. Adenosine triphosphatase (ATP)-dependent proton pump in the endosome delivers proton, within the cytoplasm, into the endosome, to lower pH in the endosome. When pH lowers, to be 5.5, the transferrin and the iron ion may separate. Then, the transferrin may return to the surface of the cell with the transferrin receptor to liberate for reuse, and the iron ion may remain within the cell to use.

As described above, the property of the blood-glucose-lowering drugs is lost after treatment for a long time, and metal intoxication is caused by directly using the metal ions for treatment, because these metal ions constantly accumulate within the body in a dipicolinate complexes type, causing side effects. Furthermore, the toxicity is reduced by combining lactoferrin with metal ion to form the complex, but the complex is decomposed in the digestive tract, such, that the structure of the complex cannot be maintained. For this reason, a complex of metal ion, for reducing the toxicity and effectively carrying the metal ion into cell, thereby encouraging glucose uptake by the cells, is provided for serving diabetics to reduce the concentration of blood glucose.

### SUMMARY

According to the foreseen problems, an object of the present invention is to a process for reducing blood glucose of a diabetic patient that reduces the toxicity and effectively carries metal ion into a cell, thereby encouraging glucose uptake by cells and serving diabetics to reduce the concentration of blood glucose.

To achieve the object, a process for reducing blood glucose of a diabetic patient includes providing a composition comprised of a complex of transferrin and at least one metal ion. A dose of the composition is administered to the diabetic patient in an amount effective to encourage glucose uptake by the cells and reduction of glucose in the blood as the transferrin carries the metal ion (or the oxidized metal ion) into the cells of the diabetic patient. Furthermore, the performance of this process has the capability of reducing insulin resistance, and therefore is available to treat high blood glucose in type 2 diabetes and reduce insulin resistance. Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given in the illustration below only, and thus is not limitative of the present invention, wherein:

FIG. 1 shows the results of the effect of a chromium (III) ion (Cr³⁺) solution on glucose absorption by C₂C₁₂ cells;

FIG. 2 shows the results of the effect of a vanadyl (VO²⁺) solution on glucose absorption by C₂C₁₂ cells;

FIG. 3 shows the results of the effect of a cobalt (III) ion (Co³⁺) solution on glucose absorption by C₂C₁₂ cells;

FIG. 4 shows the results of comparing the effects of a Cr³⁺ solution, VO²⁺ solution and Co³⁺ solutions on glucose absorption by C₂C₁₂ cells respectively;

FIG. 5 shows the results of the effect of Cr³⁺ solution with insulin on glucose absorption by C₂C₁₂ cells;

FIG. 6 shows the results of the effect of VO²⁺ solution with the insulin on glucose absorption by C₂C₁₂ cells;

FIG. 7 shows the results of the effect of Co³⁺ solution with the insulin on glucose absorption by C₂C₁₂ cells;

FIG. 8 shows the results of the effect of a complex of a transferrin and a metal ion (or an oxidized metal ion) on glucose absorption by C₂C₁₂ cells; and

FIG. 9 shows the results of the effect of the complex of the transferrin and the metal ion (or the oxidized metal ion) with the insulin on glucose absorption by C₂C₁₂ cells.

### DETAILED DESCRIPTION

The purpose, construction, features, and functions of the invention can be appreciated and understood more thoroughly through the following detailed description with reference to the attached drawings.

According to the invention, a complex for serving diabetics to reduce the concentration of blood glucose includes a transferrin and a metal ion (or an oxidized metal ion). The transferrin can carry the metal ion (or the oxidized metal ion) into cells of a patient, thereby encouraging glucose uptake by the cells and reducing the toxicity of the metal ion (or the oxidized metal ion). There are different time points for balancing the reactions of forming complexes with the transferrin and different metal ions. In general, apo-transferrin, wherein the prefix “apo-” represents a lack- state of when the transferrin released iron ion(s) is (are) bound to it. An N-terminal half molecule (i.e. N-lobe) and a C-terminal half molecule (i.e. C-lobe) of the apo-transferrin can be bound to the metal ion(s) through the binding sites of two tyrosines (Tyr), an aspartic acid (Asp), and a histidine (His). When the metal ion contacts with the N-lobe or the C-lobe, the metal ion is bound to the Tyr first. At this time, the UV absorption wavelength of another Tyr, which is not bound to the metal ion, is 274.5 nanometers (nm). If the Tyr is bound to the metal ion, the absorption value disappears at the wavelength of 274.5 nm and appears at other wavelengths. Therefore, whether the reaction is terminated can be known by measuring the absorption value.

### EXAMPLE 1

### The Complex of the Transferrin and Co³⁺

The trivalent cobalt ion (Co³⁺) solution is prepared. A cobaltous nitrate solution (Co(NO₃)₂.6H₂O) and an excess of 35% hydrogen peroxide are mixed together to form a mixture. Then, the mixture is added slowly into a sodium bicarbonate (NaHCO₃) solution (in a slurry form). The whole experiment proceeds at 0° C. for 1 hour (hr). The color of the mixture changes from brick red to olive green. An olive green product is formed and then collected. This product is a sodium cobalt carbonate compound (Na₃[Co(CO3)₃].3H₂O). Then, the Na₃[Co(CO3)₃].3H₂O dissolves in the 1 M NaHCO₃ solution to form a mixture. After the mixture remains quiet at room temperature for three days, the mixture is filtered to collect the product. The collected product is the Co³⁺ solution.

The Co³⁺ solution reacts with the transferrin. The Co³⁺ solution is mixed with the apo-transferrins with a mole ratio of 3:1 at 25° C. to form a mixture, and simultaneously the pH value is measured to keep the pH value at 7.4, where the apo-transferrins dissolve in a 0.01 M TRIS buffer containing 0.01 M NaHCO₃ (pH7.4). The absorption value of the mixture is measured at a suitable time. When the measured absorption value does not vary, the experiment can be terminated.

The surplus of Co³⁺ is removed. The complex of the transferrin and Co³⁺ and unbound Co³⁺ are separated, using a Hi-trap™ column, to acquire the complex of the transferrin and Co³⁺. The complex and Co³⁺, which have different size, are separated using the holes with different size which are formed by loading the resin to the Hi-trap™ column.

### EXAMPLE 2

### The Complex of the Transferrin and Cr³⁺

First, the trivalent chromium ion (Cr³⁺) solution is prepared. The Cr³⁺ solution reacts with the transferring referring to the method described in example 1. Finally, the surplus of Cr³⁺ is removed referring to the method described in example 1, to acquire the complex of the transferrin and Cr³⁺.

### EXAMPLE 3

### The Complex of the Transferrin and VO²⁺

First, a VO²⁺ solution is prepared. The VO²⁺ solution reacts with the transferring referring to the method described in example 1. Finally, the surplus of VO²⁺ is removed, referring to the method described in example 1, to acquire the complex of the transferrin and VO²⁺

### TEXT EXAMPLE

### Test for the Complex According to the Invention In Vitro

The cells are cultured and differentiate. In this test experiment, the C₂C₁₂ cells, which are a mouse myoblast cells, are used. The day is defined as Day 0, when C₂C₁₂ cells are cultured until the C₂C₁₂ cells reach full confluence. Then, a half of the culture media are removed and replaced with fresh differentiation media (90% DMEM (Dulbecco's Modified Eagle's Medium) and 10% FBS (fetal bovine serum)). Afterward, the culture media are replaced every 48 hrs with fresh media until the C₂C₁₂ cells differentiate from a myoblast to a myotube. In normal physiological conditions, the myotube differentiates from the myoblast and aggregates to form muscle. Therefore, the myotube has a cell type most similar to muscle, so that the cell type of used cells is a myotube in the whole test experiment.

The glucose extracted from the cells is determined. Before the start of the experiment, remove the culture media for culturing the differentiated C₂C₁₂ myotubes, wash the differentiated C₂C₁₂ myotubes with the PBS solution, and then add the culture media without the serum, to incubate for 16 hrs. When starting the experiment, add the stimulating samples with suitable concentration to the culture media and incubate at 37° C. for 30 minutes (mins). The stimulating samples, e.g. single metal ions, such as Co³⁺, Cr³⁺, or VO²⁺, transferrin, compounds of Co³⁺, Cr³⁺, or VO²⁺, insulin, etc., are mixed into DMEM without glucose. After incubation, add more 4M 2-deoxy-D-glucose (2DG), and then incubate at 3 7C for 10 mins. Finally, incubate the cells on ice for 10 mins, to stop the reaction.

Then, remove the culture media, and wash the cells three times with an ice-cold PBS solution. After washing, scrape the cells from the plate with ice-cold 65% EtOH and sonicate the cells. After spun in a centrifuge at 13000 rpm and 4° C. for 15 mins, transfer the supernatant to fresh the eppendorf tube and then spin the remainder in the centrifuge again to collect the supernatant again. The collected supernatants are mixed and dry the supernatant by vacuum centrifugation. After drying, the sample is acquired and assayed for glucose oxidase levels using the Amplex Red Glucose/Glucose Oxidase Assay Kit.

### TEXT EXAMPLE

### Result and Discussion

1. Control Groups in Contrast to Test Group According to the Invention: the Effect of Single Metal Ions on Glucose Absorption by C₂C₁₂ Myotubes

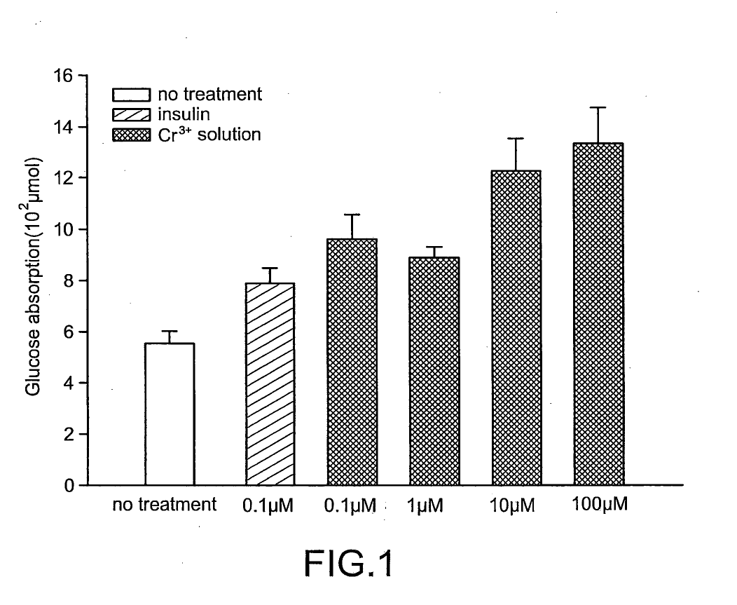

Before the experiment, the cells differentiated from the C₂C₁₂ myoblasts to the C₂C₁₂ myotubes and the culture media are replaced with the culture media without the serum to incubate more for 16 hrs. Afterwards, the C₂C₁₂ myotubes are stimulated by a different metal solution to observe the effect on glucose absorption. FIG. 1 shows the results of the effect of the Cr³⁺ solution on the glucose absorption by the C₂C₁₂ cells (i.e. the C₂C₁₂ myotubes). Referring to FIG. 1, when the C₂C₁₂ cells are stimulated by Cr³⁺, where the concentrations of the Cr³⁺ solutions are 0.1 μM, 1 μM, 10 μM, and 100 μM respectively,. their glucose absorptions are enhanced in contrast to the C₂C₁₂ cells without stimulation by the metal solution. In contrast to the C₂C₁₂ cells without stimulation by the metal solution, the capabilities of enhancing the glucose absorptions by the C₂C₁₂ cells stimulated with 10 μM, and 100 μM Cr³⁺ solution are the best. There are 2.21 fold and 2.41 fold increases in the glucose absorptions respectively.

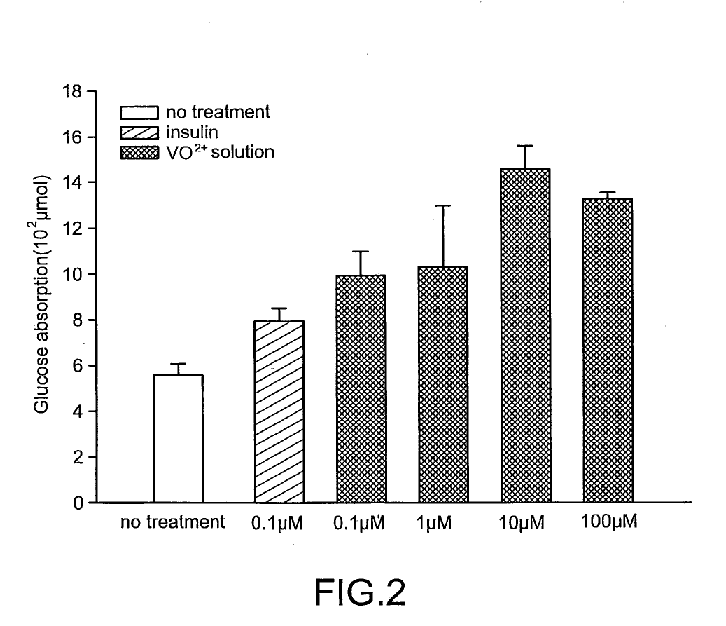

FIG. 2 shows the results of the effect of the VO²⁺ solution on the glucose absorption by the C₂C₁₂ cells (i.e. the C₂C₁₂ myotubes). Referring to FIG. 2, when the C₂C₁₂ cells are stimulated by VO²⁺, where the concentrations of the VO²⁺ solutions are 0.1 μM, 1 μM, 10 μM, and 100 μM respectively, their glucose absorptions are enhanced in contrast to the C₂C₁₂ cells without stimulation by the metal solution. In contrast to the C₂C₁₂ cells without stimulation by the metal solution, the capabilities of enhancing the glucose absorptions by the C₂C₁₂ cells stimulated with 10 μM, and 100 μM VO²⁺ solution are the best, and there are 2.61 fold and 2.38 fold increases in the glucose absorptions respectively.

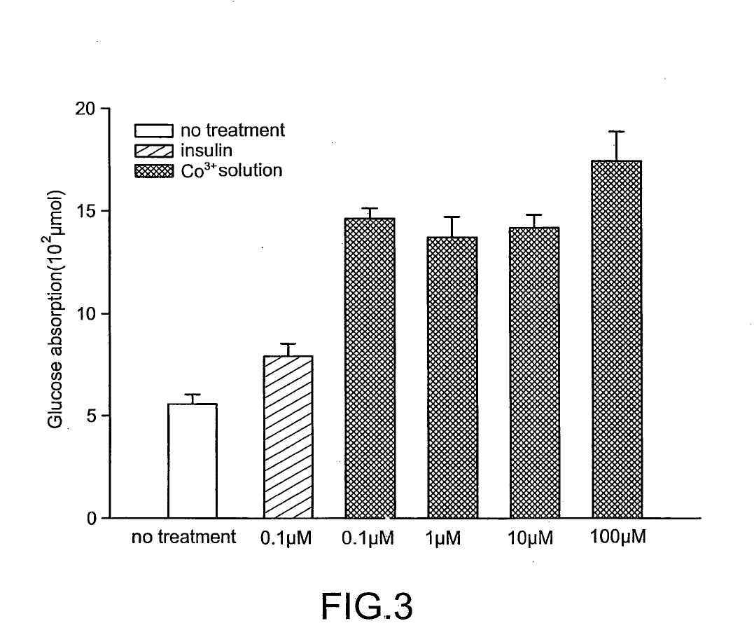

FIG. 3 shows the results of the effect of the Co³⁺ solution on the glucose absorption by the C₂C₁₂ cells (i.e. the C₂C₁₂ myotubes). Referring to FIG. 3, when the C₂C₁₂ cells are stimulated by Co³⁺, where the concentrations of the Co³⁺ solutions are 0.1 μM, 1 μM, 10 μM, and 100 μM respectively, their glucose absorptions are enhanced in contrast to the C₂C₁₂ cells without stimulation by the metal solution. In contrast to the C₂C₁₂ cells without stimulation by the metal solution, the result of enhancing the glucose absorption by the C₂C₁₂ cells stimulated with 100 μM Co³⁺ solution is the best, and there is a 3.14 fold increase in the glucose absorption. However, the type of C₂C₁₂ cells changes and the C₂C₁₂ cells float easily after the C₂C₁₂ cells are stimulated with the Co³⁺ solution for 30 mins. Thus it can be seen that the C₂C₁₂ cells are damaged at a certain level, although the glucose absorption by the C₂C₁₂ cells, stimulated with 100 M Co³⁺ solution, is the best.

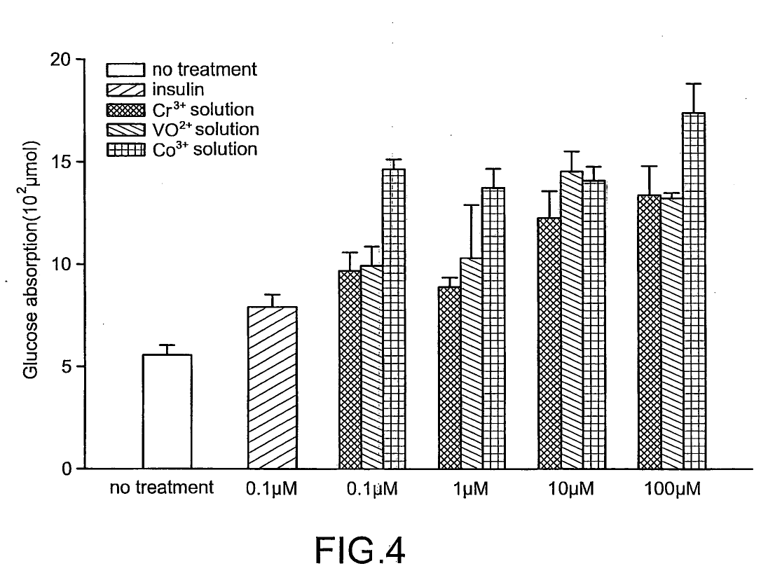

FIG. 4 shows the results of comparing the effects of the Cr³⁺ solution, VO²⁺ solution and Co³⁺ solution on the glucose absorptions by the C₂C₁₂ cells (i.e. the C₂C₁₂ myotubes) respectively. In FIG. 4, the columns with the same patterns represent the C₂C₁₂ cells stimulated by the same metal ion (or oxidized metal ion) solutions and the sign under the transverse axle indicates the concentration of the metal ion (or oxidized metal ion) solution, corresponding to each column. Referring to FIG. 4, the result of enhancing the glucose absorption by the C₂C₁₂ cells stimulated with 100 μM Co³⁺ solution is the best, and the results of stimulations with 100 μM Cr³⁺ solution and 100 μM VO²⁺ solution are reached by stimulation with 0.1 μM Co³⁺ solution. However, the type of C₂C₁₂ cells changes and the C₂C₁₂ cells float easily after the C₂C₁₂ cells are stimulated with the Co³⁺ solution for 30 mins. Thus it can be seen that the C₂C₁₂ cells are damaged at a certain level, although the glucose absorption by the C₂C₁₂ cells stimulated with 100 μM Co³⁺ solution is the best. As a whole, the result of enhancing the glucose absorption by the C₂C₁₂ cells stimulated with the Co³⁺ solution is the best, but the C₂C₁₂ cells are damaged by the Co³⁺ solution with high concentration.

Further, when some blood-glucose-lowering substances are used to treat with insulin, the glucose absorption by the C₂C₁₂ cells is enhanced. For this reason the effects on the glucose absorptions by the C₂C₁₂ cells when the Cr³⁺ solution, VO²⁺ solution and Co³⁺ solution are used to stimulate the C₂C₁₂ cells with the insulin respectively, are studied.

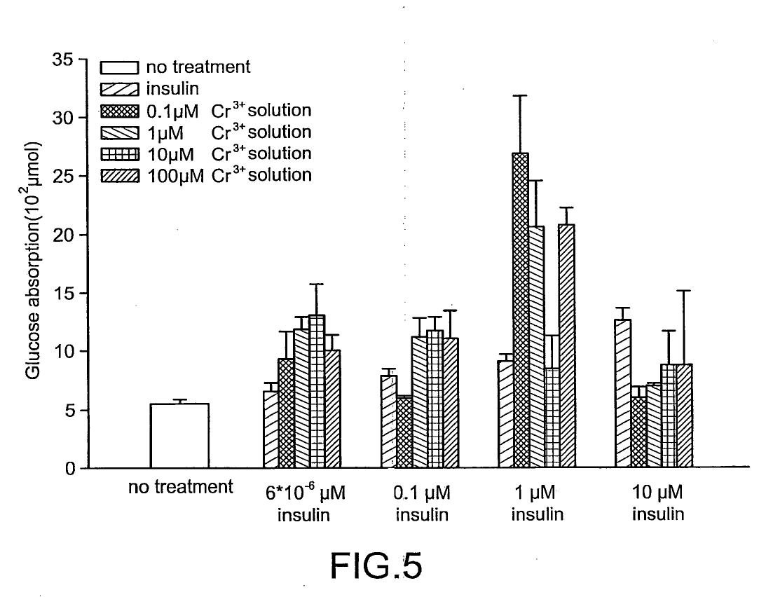

FIG. 5 shows the results of the effect of the Cr³⁺ solution with insulin on the glucose absorption by the C₂C₁₂ cells (i.e. the C₂C₁₂ myotubes), where the columns with the same patterns represent the C₂C₁₂ cells stimulated by the Cr³⁺ solutions with the same concentration and the sign under the transverse axle indicates the concentration of the insulin corresponding to each column. As shown in FIG. 5, under the concentration of 0.1 μM (Cr³⁺ solution), the result of co-treatment with 1 μM insulin is the best, and there are 4.84 fold increases in the glucose absorption in contrast to the result without treatment with any stimulating sample. Under the concentration of 1 μM (Cr³⁺ solution), the result of co-treatment with 1 μM insulin is the best, and there are 3.71 fold increases in the glucose absorption in contrast to the result without treatment with any stimulating sample. Under the concentration of 10 μM (Cr³⁺ solution), the result of co-treatment with 6 pM .(i.e. 6×10⁻⁶ μM) insulin is the best, and there are 2.36 fold increases in the glucose absorption in contrast to the result without treatment with any stimulating sample. Under the concentration of 100 μM (Cr³⁺ solution), the result of co-treatment with 1 μM insulin is the best, and there are 3.74 fold increases in the glucose absorption in contrast to the result without treatment with any stimulating sample.

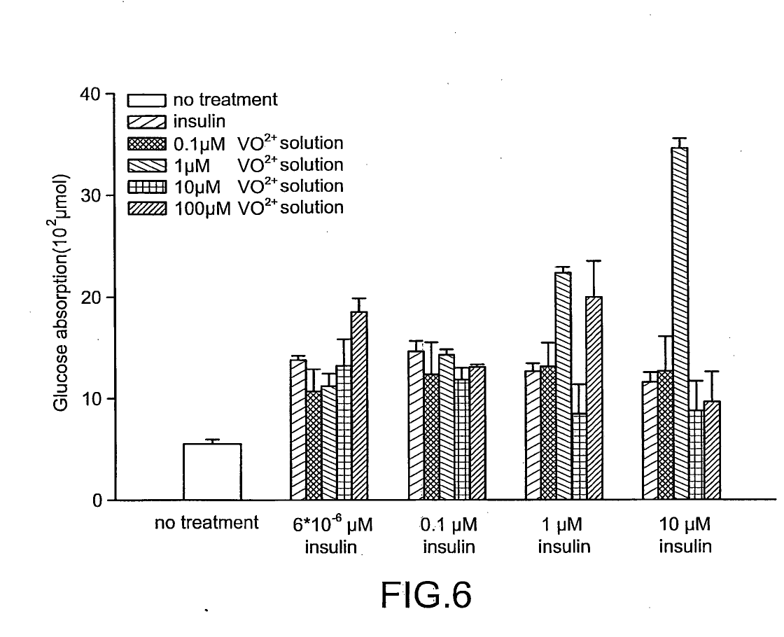

FIG. 6 shows the results of the effect of the VO²⁺ solution with insulin on the glucose absorption by the C₂C₁₂ cells (i.e. the C₂C₁₂ myotubes), where the columns with the same patterns represent the C₂C₁₂ cells stimulated by the VO²⁺ solutions with the same concentration, and the sign under the transverse axle indicates the concentration of the insulin corresponding to each column. As shown in FIG. 6, under the concentration of 0.1 μM (VO²⁺ solution), the result of co-treatment with 0.1 μM insulin is the best, and there are 2.62 fold increases in the glucose absorption in contrast to the result without treatment with any stimulating sample. Under the concentration of 1 μM (VO²⁺ solution), the result of co-treatment with 10 μM insulin is the best, and there are 2.7 fold increases in the glucose absorption in contrast to the result without treatment with any stimulating sample. Under the concentration of 10 μM (VO²⁺ solution), the result of co-treatment with 10 μM insulin is the best, and there are 6.21 fold increases in the glucose absorption in contrast to the result without treatment with any stimulating sample. Under the concentration of 100 μM (VO²⁺ solution), the result of co-treatment with 1 μM insulin is the best, and there are 3.4 fold increases in the glucose absorption in contrast to the result without treatment with any stimulating sample.

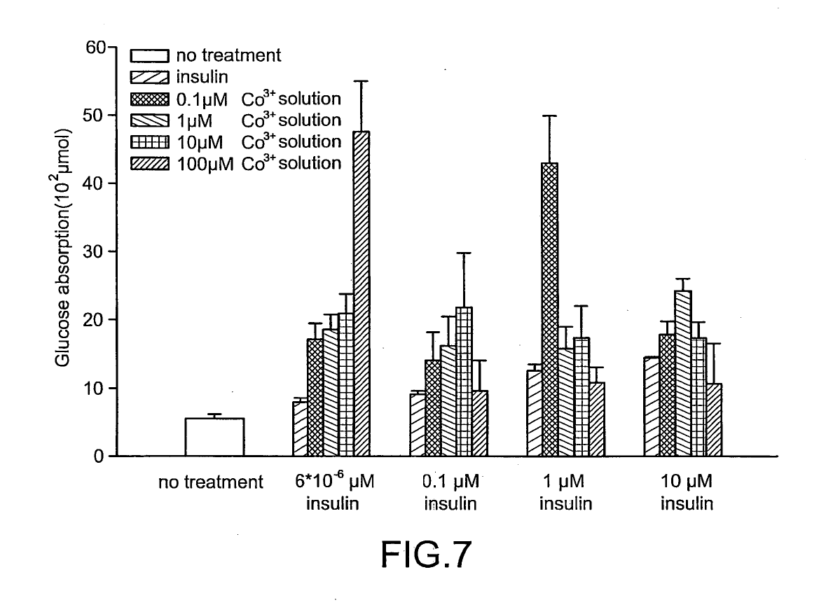

FIG. 7 shows the results of the effect of the Co³⁺ solution with insulin on the glucose absorption by the C₂C₁₂ cells (i.e. the C₂C₁₂ myotubes), where the columns with the same patterns represent the C₂C₁₂ cells stimulated by the Co³⁺ solutions with the same concentration and the sign under the transverse axle indicates the concentration of the insulin corresponding to each column. As shown in FIG. 7, under the concentration of 0.1 μM (Co³⁺ solution), the result of co-treatment with 1 μM insulin is the best, and there are 7.73 fold increases in the glucose absorption in contrast to the result without treatment with any stimulating sample. Under the concentration of 1 μM (Co³⁺ solution), the result of co-treatment with 10 μM insulin is the best, and there are 4.37 fold increases in the glucose absorption in contrast to the result without treatment with any stimulating sample. Under the concentration of 10 μM (Co³⁺ solution), the result of co-treatment with 6 pM insulin is the best, and there are 3.95 fold increases in the glucose absorption in contrast to the result without treatment with any stimulating sample. Under the concentration of a 100 μM (Co³⁺ solution), the result of co-treatment with 1 μM insulin is the best, and there is 8.5 fold increases in the glucose absorption in contrast to the result without treatment with any stimulating sample. But the type of C₂C₁₂ cells changes and the C₂C₁₂ cells are damaged, such as that, cells float easily, etc., under the concentration of 100 μM (Co³⁺ solution).

Based on above experiments, the capability of enhancing the glucose absorptions by the myocytes is certainly enhanced by the metal ion (or oxidized metal ion), wherein the result of treatment with Co³⁺ solution is the best. But, the type of the C₂C₁₂ cells changes and the C₂C₁₂ cells float easily under treatment with 100 μM Co³⁺ solution. Furthermore, when the metal ions (or oxidized metal ions) are co-treated with insulin, the glucose absorption by the C₂C₁₂ cells is better than the glucose absorption after treatment with single metal ions (or oxidized metal ions). Herein, the result of co-treatment of the Co³⁺ solution and the insulin is the best, but the type of C₂C₁₂ cells changes under treatment with a 100 μM Co³⁺ solution.

2. Test Group According to the Invention: the Effect of the Complex of the Transferrin and the Metal Ion (or an Oxidized Metal Ion) on Glucose Absorption by C₂C₁₂ Myotubes

As the above, it is known that the glucose absorption by the C₂C₁₂ cells enhanced by the 100 μM Co³⁺ solution is the best, but the. type of the C₂C₁₂ cells changes and the C₂C₁₂ cells are damaged. This phenomenon is improved using the complex of the transferrin and the metal ion (or an oxidized metal ion) according to the invention. In the organism, the complex of the transferrin and the iron ion can bind to a transferrin receptor on cell membrane, and then enter into the cell by the endocytosis to form endosome. An adenosine triphosphatase (ATP)-dependent proton pump in the endosome delivers the proton within the cytoplasm into the endosome, to lower the pH in the endosome. When the pH lowers to be 5.5, the transferrin and the iron ion, which are bound, separate. Then, the transferrin returns the surface of the cell with the transferrin receptor to be liberated for reuse, and the iron ion may remain within the cell to use. Finally, a group of iron ions aggregate to form a ferritin, and then the ferritin is decomposed by lysosome. The lobe of the transferrin is general in open state, but sometimes in closed state, which might occur by mere chance. The iron (III) ion (Fe³⁺) and carbonic acid (CO₃²⁻) are bound to two tyrosines (Tyr) of domain 2 of the transferrin, so that the transferrin is in closed state. Then, Fe³⁺ and CO₃²⁻ are bound to one aspartic acid (Asp), and one histidine (His) more, so that the transferrin maintains in closed state. In the transferrin, there is a release mechanism for the Fe³⁺, that is, each of the domains 1 and 2 of an N-terminal half molecule (i.e. N-lobe) has a lysine. When the N-lobe of the transferrin is in closed state, two lysines do not repel, to be in a stable closed state. When pH lowers, two lysines have positive charges and repel, so the N-lobe of the transferrin is in open state to release the Fe³⁺. For the complex according to the invention, the transferrin is transferred into the cells by the metal ion (or the oxidized metal ion) depending on above mechanism. Therefore the glucose absorption by the C₂C₁₂ cells is improved by the metal ion (or the oxidized metal ion). In the prior art, the complex compound of lactoferrin and metal ion is provided for treating diabetes, as shown in the U.S. Pat. No. 6,379,693. But, in general, the surface of somatic or body cells, such as myocytes, do not have a lactoferrin receptor, so the complex compound of lactoferrin cannot enter into the cells by the endocytosis. However, the transferrins are a large number of proteins in the blood of the human body, and the myocytes in the human body generally have a transferrin receptor. Therefore, the complex of the transferrin and the metal ion (or an oxidized metal ion) certainly enters into the cell by the endocytosis in the circulatory system. Moreover, since the transferrins are the proteins in the blood of the human body, the side effects, such as immunoreaction, etc., are avoided while treating.

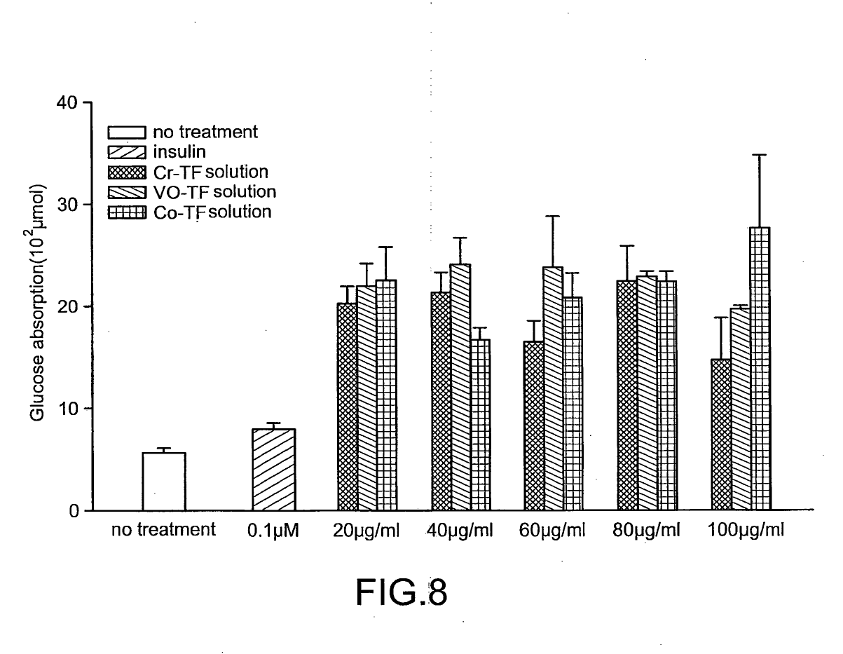

FIG. 8 shows the results of the effect of the complex of the transferrin and the metal ion (or the oxidized metal ion) on the glucose absorption by the C₂C₁₂ cells (i.e. the C₂C₁₂ myotubes). In FIG. 8, the columns with the same patterns represent treatment with the same complex and the sign under the transverse axle indicates the concentration of the complex corresponding to each column. As shown in FIG. 8, in contrast to the result without treatment with any stimulating sample, the glucose absorption by the C₂C₁₂ cells treated with 80 micrograms per milliliter (μg/ml) complex of the transferrin and Cr³⁺ (Cr-TF) can increase 4.03 fold, the glucose absorption by the C₂C₁₂ cells treated with 40 μg/ml complex of the transferrin and VO²⁺ (VO-TF) can increase 4.43 fold, and the glucose absorption by the C₂C₁₂ cells treated with 100 μg/ml complex of the transferrin and Co³⁺ (Co-TF) can increase 4.97 fold. Comparing the result shown in FIG. 8 with the results in FIGS. 1, 2, and 3, the result of improving the glucose absorption by the C₂C₁₂ cells: using combining the metal ion (or the oxidized metal ion) with the transferrin to provide a bio-carrying capability is better than that using a single metal ion (or the oxidized metal ion). Moreover, under treatment with the complex of the transferrin and Co³⁺ (Co-TF), the change in type of cells and the damage to cells are avoided.

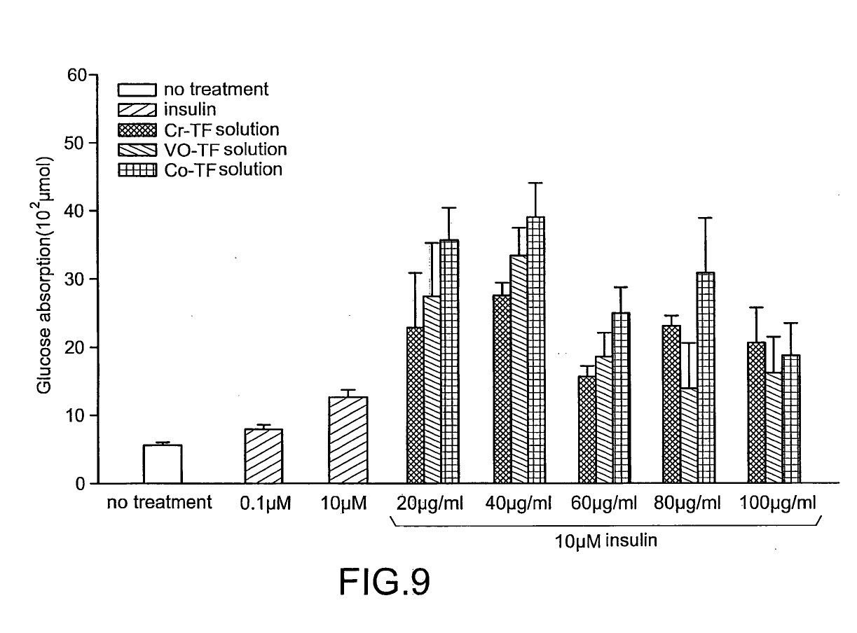

FIG. 9 shows the results of the effect of the complex of the transferrin and the metal ion (or the oxidized metal ion) with the insulin on glucose absorption by C₂C₁₂ cells (i.e. the C₂C₁₂ myotubes). In FIG. 9, the columns with the same patterns represent treatment with the same complex, the sign under the transverse axle indicates the concentration of the complex corresponding to each column, and the sign under each sign of the concentration of the complex indicates the concentration of the insulin corresponding to each column. As shown in FIG. 9, in contrast to the result without treatment with any stimulating sample, the glucose absorption by the C₂C₁₂ cells co-treated with 40 μg/ml complex of the transferrin and Cr³⁺ (Cr-TF) and the 10 μM insulin can increase 4.94 fold, the glucose absorption by the C₂C₁₂ cells co-treated with 40 μg/ml complex of the transferrin and VO²⁺ (VO-TF) and the 10 μM insulin can increase 6.07 fold, and the glucose absorption by the C₂C₁₂ cells co-treated with 40 μg/ml complex of the transferrin and Co³⁺ (Co-TF) and the 10 μM insulin can increase 7.01 fold.

Compared the result under treatment of the complex of the transferrin and metal ion (or the oxidized metal ion) to that under treatment of a single metal ion (or the oxidized metal ion), the results are as follows. For the Cr³⁺, the glucose absorption by the C₂C₁₂ cells treated with 1 mM Cr³⁺ solution can increase 1.6 fold, and the glucose absorption by the C₂C₁₂ cells treated with 40 milligrams per milliliter (mg/ml) Cr³⁺ solution including 1 mM Cr³⁺ can increase 3.9 fold. For the VO²⁺, the glucose absorption by the C₂C₁₂ cells treated with 1 mM VO²⁺ solution can increase 1.9 fold, and the glucose absorption by the C₂C₁₂ cells treated with 40 mg/ml VO²⁺ solution including 1 mM VO²⁺ can increase 4.4 fold. For the Co³⁺, the glucose absorption by the C₂C₁₂ cells treated with 1 mM Co³⁺ solution can increase 2.5 fold, and the glucose absorption by the C₂C₁₂ cells treated with 40 mg/ml Co³⁺ solution including 1 mM Co³⁺ can increase 3.0 fold. By contrast, under the glucose absorption by myocytes, the result of stimulating the myocytes by the complex of the transferrin and the metal ion (or the oxidized metal ion) is better than the result of stimulating the myocytes by a single metal ion (or the oxidized metal ion).

Comparing the result of stimulating the myocytes by the complex of the transferrin and metal ion (or the oxidized metal ion) and the insulin with that by single metal ion (or the oxidized metal ion) and the insulin, the results are as follows. For the Cr³⁺, the glucose absorption by the C₂C₁₂ cells treated with 1 mM Cr³⁺ solution and the insulin can increase 1.3 fold, and the glucose absorption by the C₂C₁₂ cells treated with 40 mg/ml Cr-TF solution including 1 mM Cr³⁺ and the insulin can increase 5.0 fold. For the VO²⁺, the glucose absorption by the C₂C₁₂ cells treated with 1 mM VO²⁺ solution and the insulin can increase 2.7 fold, and the glucose absorption by the C₂C₁₂ cells treated with 40 mg/ml VO-TF solution including 1 mM VO²⁺ and insulin can increase 6.1 fold. For the Co³⁺, the glucose absorption by the C₂C₁₂ cells treated with 1 mM Co³⁺ solution and the insulin can increase 4.4 fold, and the glucose absorption by the C₂C₁₂ cells treated with 40 mg/ml Co-TF solution and the insulin including 1 mM Co³⁺ can increase 7.1 fold. By contrast, when the insulin exists, under stimulating the glucose absorption by myocytes, the result of stimulating the myocytes with the complex of the transferrin and the metal ion (or the oxidized metal ion) is better than the result of stimulating the myocytes with a single metal ion (or the oxidized metal ion).

Based on above experiments, under the glucose absorptions by the myocytes, the result of stimulation with the complex of the transferrin and the metal ion (or the oxidized metal ion) is better than the result of stimulation with a single metal ion (or an oxidized metal ion). Moreover, the glucose absorption by the myocytes is more enhanced while co-treating with a definite amount of insulin. Therefore, the glucose absorption by the myocytes can be enhanced using the complex of the transferrin and the metal ion (or the oxidized metal ion) according to the invention, that is, the blood glucose can be reduced under physiology, thereby treating diabetes. The glucose uptake by the cells can be encouraged using the complex of the transferrin and the metal ion (or the oxidized metal ion) according to the invention, that is, the concentration of glucose within the body. fluid of the peripheral tissue/organ can be reduced under physiology, and the toxicity of the metal ion (or the oxidized metal ion) can be reduced.

Knowing the invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A process for reducing blood glucose of a diabetic patient, comprising the steps of:
providing a composition comprised of a complex of transferrin and at least one metal ion; and
administering a dose of the composition to the diabetic patient in an amount effective to encourage glucose uptake by the cells and reduction of glucose in the blood as the transferrin carries the at least one metal ion into the cells of the diabetic patient.

2. The process of claim 1, wherein the metal ion is a divalent metal ion.

3. The process of claim 1, wherein the metal ion is a trivalent metal ion.

4. The process of claim 1, wherein the metal ion is a trivalent chromium ion (Cr³⁺).

5. The process of claim 1, wherein the metal ion is a trivalent cobalt ion (Co³⁺).

6. The process of claim 1, wherein the metal ion is an oxidized metal ion.

7. The process of claim 6, wherein the oxidized metal ion is a vanadyl (VO²⁺).
